(19) United States
(12) Patent Application Publication
(10) Pub. No.: US 2025/0186234 A1
(43) Pub. Date: Jun. 12, 2025

(54) ARM SLEEVE THAT PROVIDES ASSISTANCE DURING AN ARM MOTION

(71) Applicant: Jason Colleran, Roswell, GA (US)

(72) Inventor: Jason Colleran, Roswell, GA (US)

(21) Appl. No.: 18/977,855

(22) Filed: Dec. 11, 2024

Related U.S. Application Data
(60) Provisional application No. 63/608,862, filed on Dec. 12, 2023.

(51) Int. Cl. A61F 5/01

(52) U.S. Cl. CPC A61F 5/0118; A61F 2005/0197

(57) ABSTRACT

The arm sleeve provides external assistance to the arm and shoulder of a user during an arm motion, such as a throwing motion. An example arm sleeve includes a framework configured to assist at least one muscle supporting the ulnar collateral ligament (UCL) of the user's arm and at least one muscle supporting the glenohumeral joint of the user's shoulder during the arm motion. The framework comprises a plurality of anchor plates and elastic bands.

### CROSS-REFERENCE TO RELATED APPLICATION(S)

This application claims the benefit of U.S. Provisional Application Ser. No. 63/608,862, filed on Dec. 12, 2023, the entirety of which is incorporated herein by reference.

### TECHNICAL FIELD

This disclosure relates to implementations of an arm sleeve that provides external assistance to the arm and shoulder during an arm motion.

### BACKGROUND

An arm motion, such as an overhead throwing motion when pitching a baseball, generally involves a winding-up motion, acceleration of the arm, release of the ball, and follow-through. During acceleration and follow-through, valgus and varus torque are generated, placing valgus and varus forces on the elbow joint, particularly on the soft medial structures of the elbow, including the medial collateral ligament, also known as the ulnar collateral ligament (UCL).

Placing large valgus and varus loads, such as those generated by repeated hard throwing or pitching, on the elbow can lead to injury of the UCL, including overstretching, fraying, and tearing. The UCL may be injured either by acute ligament tears or through overuse and repetitive stress. For example, repeated valgus and varus stresses from repetitive throwing may result in overuse injuries where tissue breakdown exceeds the tissue's ability to repair itself. In children, such repetitive stress may manifest as “little league elbow,” which can lead to medial epicondylar apophysitis and stress fractures of the medial epicondylar epiphyses.

Injury to the UCL can be particularly problematic because the UCL, along with the lateral collateral ligament, serves as a primary source of stability for the elbow. When the UCL is damaged or injured and does not heal correctly, the elbow can become loose and/or unstable, leading to further injury. This may result in the need for corrective surgery, such as Tommy John surgery, to repair the UCL.

In general, previous attempts at preventative or rehabilitation devices or garments have focused on either compression, such as a tight-fitting sleeve over the elbow, or braces designed to provide support and/or limit arm motion. However, these prior approaches have focused on providing structural support for the elbow, rather than on providing positive assistance to the muscles in the arm and shoulder, which help stabilize the UCL and thereby reduce valgus and varus loads.

Accordingly, there exists a need for the arm sleeve disclosed herein. The present invention is directed to an arm sleeve configured to provide external assistance to the arm and shoulder during an arm motion, addressing these and other needs.

### SUMMARY OF THE INVENTION

It is to be understood that this summary is not an extensive overview of the disclosure. This summary is exemplary and not restrictive, and it is intended neither to identify key or critical elements of the disclosure nor delineate the scope thereof. The sole purpose of this summary is to explain and exemplify certain concepts of the disclosure as an introduction to the following complete and extensive detailed description.

Implementations of an arm sleeve that provides external assistance to an arm and shoulder of a user during an arm motion, such as a throwing motion, are disclosed. The arm sleeve is configured to decrease elbow joint forces present during the end-range cocking phase of an overhead throwing motion. Specifically, the arm sleeve reduces the distraction force on the medial side of the elbow and the compressive force on the lateral side of the elbow. Additionally, the arm sleeve is configured to assist with the initiation of the acceleration phase of an overhead throwing motion by providing elastic assistance to the shoulder joint. Furthermore, the arm sleeve provides elastic resistance during the deceleration and follow-through phases of an overhead throwing motion, reducing the eccentric contraction demands of the shoulder's external rotators. The arm sleeve is also configured to assist with transitioning the forearm and hand from supination to pronation during a throwing motion.

An example arm sleeve comprises: a torso portion configured to be attached to the torso of the user, an arm portion configured to cover at least a portion of the arm of the user, and a framework comprising a plurality of anchor plates and elastic bands. The plurality of anchor plates includes a first anchor plate; the first anchor plate is attached to the torso portion and extends from a front side of the torso portion, across a top of the shoulder of the user, to a back side of the torso portion. The plurality of anchor plates includes a second anchor plate; the second anchor plate is attached to the arm portion and is positioned to overlay at least a portion of the triceps and deltoid of the arm of the user. The plurality of anchor plates includes a third anchor plate; the third anchor plate is attached to the arm portion and is positioned to partially encircle a forearm of the arm when the arm sleeve is being worn. The elastic bands include a first elastic band with a first end that extends from a portion of the first anchor plate overlaying the front side of the torso portion to a second end attached to the second anchor plate. The elastic bands include a second elastic band with a first end that extends from a portion of the first anchor plate overlaying the shoulder of the user to a second end attached to the second anchor plate. The elastic bands include a third elastic band with a first end that extends from a portion of the first anchor plate overlaying the back side of the torso portion to a second end attached to the second anchor plate. The elastic bands include a fourth elastic band with a first end that extends from the second anchor plate across the triceps area of the arm, past the elbow joint, and posteriorly to a second end that is attached to the third anchor plate. The elastic bands include a fifth elastic band with a first end that extends from the second anchor plate, spirals posteriorly across the triceps area of the arm, past the elbow joint, and continues anteriorly to a second end that is attached to the third anchor plate. The first anchor plate, the second anchor plate, and the third anchor plate each serve as anchor points for one or more of the elastic bands of the framework.

In some implementations, the elastic bands of the arm sleeve includes a sixth elastic band with a first end that extends from the second anchor plate, spirals posteriorly across the triceps area of the arm, past the elbow joint, and continues anteriorly to a second end that is attached to the third anchor plate.

### BRIEF DESCRIPTION OF THE DRAWINGS

FIG. 1 is a front view of an arm sleeve that provides assistance during an arm motion; wherein the arm sleeve is shown being worn.

FIG. 2 is a side view of the arm sleeve shown in FIG. 1.

FIG. 3 is a rear view of the arm sleeve shown in FIG. 1.

FIG. 4 is a front elevational view of the arm sleeve shown in FIG. 1; wherein the arm sleeve is lying flat and the torso strap has been removed for clarity of disclosure.

FIG. 5 is a rear elevational view of the arm sleeve shown in FIG. 4.

FIG. 6 is a top plan view of the arm sleeve shown in FIG. 4.

FIG. 7 is a bottom plan view of the arm sleeve shown in FIG. 4.

FIG. 8 is a left side elevational view of the arm sleeve shown in FIG. 4.

FIG. 9 is a right side elevational view of the arm sleeve shown in FIG. 4.

FIG. 10 is a front elevational view of the torso strap, shown in FIG. 1, used to position the arm sleeve on the torso of the person. A symbolic break in the length is represented by a centrally located pair of adjacent lines.

FIG. 11 is a rear elevational view of the torso strap shown in FIG. 10.

FIG. 12 is a side elevational view of the torso strap shown in FIG. 10.

Like reference numerals refer to corresponding parts throughout the several views of the drawings.

### DETAILED DESCRIPTION

An arm motion, such as an overhead throwing motion when pitching a baseball, generally involves a winding-up motion, an acceleration of the arm, release of the ball, and a follow-through. Other similar arm motions may include a swinging motion, such as tennis, handball, volleyball, etc. that generally involve a winding-up motion, an acceleration of the arm, hitting the ball, and a follow-through. When the arm is accelerating and following through, valgus and varus torque are generated, thereby placing a valgus and a varus force on the elbow joint, in particular, the soft medial structures of the elbow including the medial collateral ligament, otherwise known as the ulnar collateral ligament (UCL).

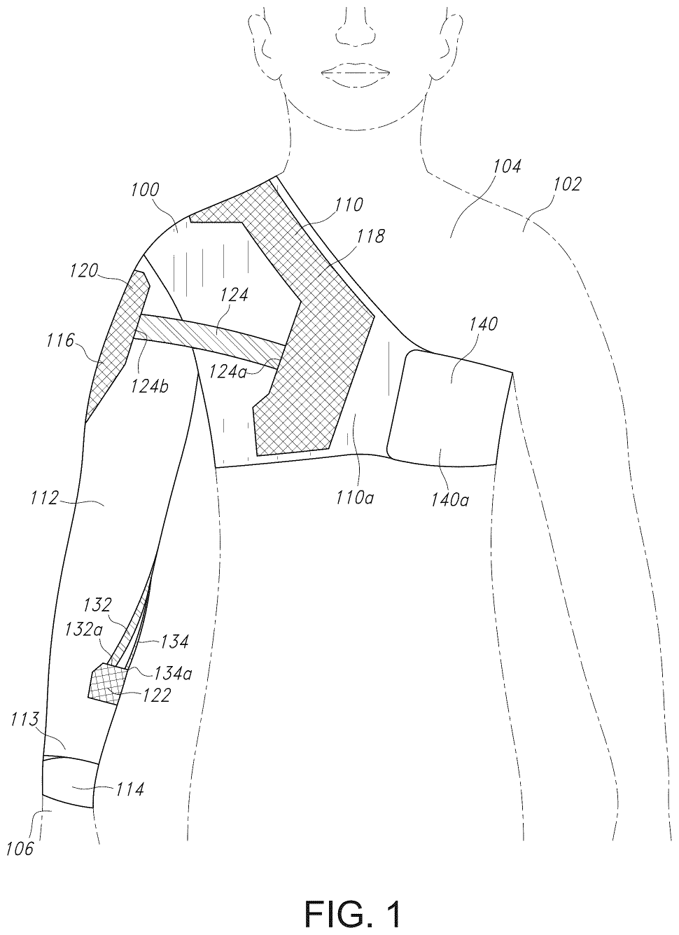

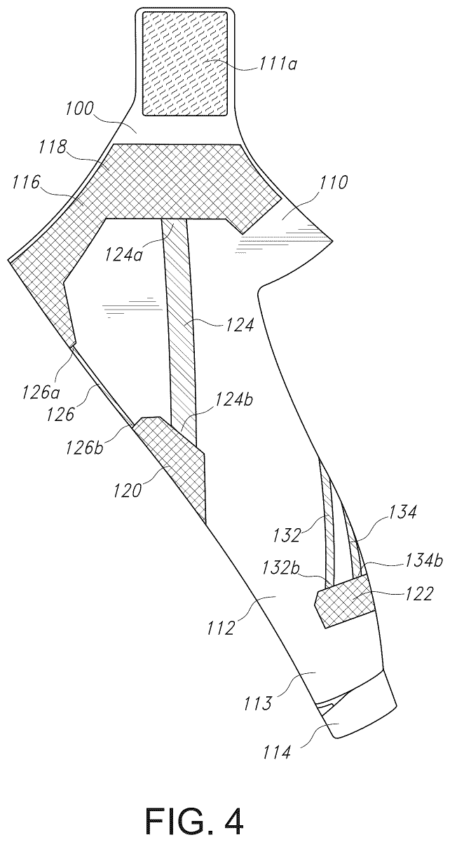

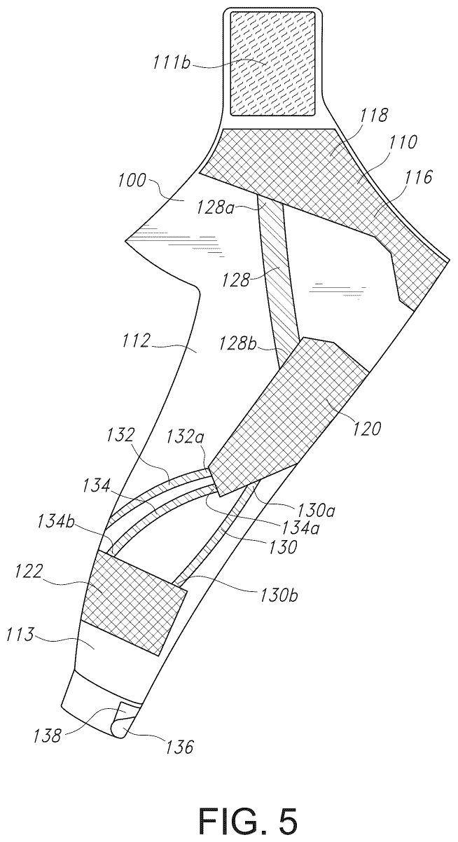

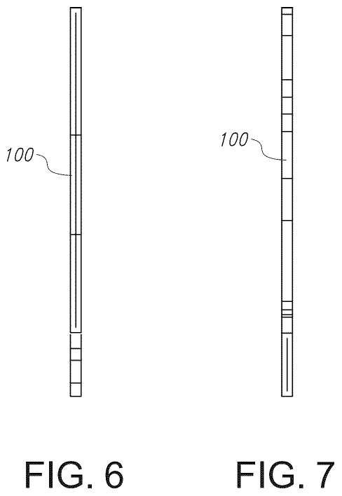

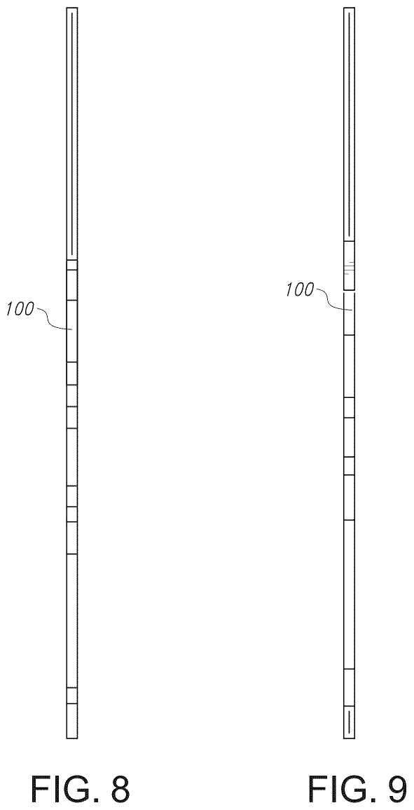

FIGS. 1-9 illustrate an example arm sleeve 100 according to the principles of the present disclosure. The arm sleeve 100 is configured to decrease elbow joint forces present during the end-range cocking phase of an overhead throwing motion. Specifically, the arm sleeve 100 reduces the distraction force on the medial side of the elbow and the compressive force on the lateral side of the elbow. Additionally, the arm sleeve 100 is configured to assist with the initiation of the acceleration phase of an overhead throwing motion by providing elastic assistance to the shoulder joint. Furthermore, the arm sleeve 100 provides elastic resistance during the deceleration and follow-through phases of an overhead throwing motion, reducing the eccentric contraction demands of the shoulder's external rotators. The arm sleeve 100 is also configured to assist with transitioning the forearm and hand from supination to pronation during a throwing motion.

As shown in FIGS. 1-3, the arm sleeve 100 is configured to be worn on the throwing arm 106 of a user 102. While the embodiment of the arm sleeve 100 shown in FIGS. 1-3 is configured to be worn on the right arm 106, the arm sleeve 100 may be configured and adapted to be worn on the left arm if desired.

As shown in FIGS. 1-5, the arm sleeve 100 includes a torso portion 110 configured to be attached to the torso 104 of a user 102 and an arm portion 112 configured to cover at least a portion of the throwing arm 106 of the user 102 while the arm sleeve 100 is being worn. In the implementation shown in FIGS. 1-3, the arm portion 112 comprises a sleeve that extends below the elbow joint of the user's throwing arm 106 to the wrist, and a wrist strap 114 configured to secure the distal end of the arm portion 112 about the wrist of the user's throwing arm 106. However, in other implementations, the arm portion 112 may be shorter and not extend to the user's wrist. Additionally, in some implementations, the arm portion 112 may omit the wrist strap 114.

The torso portion 110 and the arm portion 112 of the arm sleeve 100 may be made of a stretchable fabric, such as spandex, or any other suitable material that allows for a snug fit when the arm sleeve 100 is worn. Accordingly, the material of the torso portion 110 and arm portion 112 may be relatively elastic, as desired, to allow for the snug fit. Additionally, in some implementations, the torso portion 110 of the arm sleeve 100 may comprise a different material, different thickness of material, different density of material, or different elasticity than the arm portion 112 of the arm sleeve 100. Thus, as a non-limiting example, the torso portion 110 and the arm portion 112 may comprise a single piece of material such as spandex. Additionally, the torso portion 110 and/or the arm portion 112 may include cut-outs, slits, etc. to allow for moisture and/or air ventilation as desired.

As shown in FIGS. 1-5, the arm sleeve 100 includes a framework 116 configured to provide assistance to at least one muscle supporting an ulnar collateral ligament (UCL) of the arm 106 of the user 102 during an arm motion. Additionally, the framework 116 is also configured to provide assistance to at least one muscle supporting the glenohumeral joint of the shoulder of the user 102 during an arm motion. The framework 116 comprises a plurality of anchor plates (118, 120, 122) and elastic bands (124, 126, 128, 130, 132, 134).

As shown best in FIGS. 1-3, in some implementations, the framework 116 of the arm sleeve 100 includes three anchor plates (118, 120, 122). One anchor plate 118 is attached to the torso portion 110 and extends from the front side 110a of the torso portion 110, across the top of the shoulder of the throwing arm 106, to the back side 110b of the torso portion 110. The other two anchor plates (120, 122) are attached to the arm portion 112. One of these anchor plates 120 is positioned to overlay at least a portion of the triceps and deltoid of the throwing arm 106, and the other anchor plate 122 is positioned to partially encircle the forearm of the throwing arm 106, above the wrist. The anchor plates (118, 120, 122) are configured to conform to the general area of the body that they overlay and to serve as anchor points for the elastic bands (124, 126, 128, 130, 132, 134) of the framework 116. The anchor plates (118, 120, 122) are made of a rigid material, such as non-elastic thermoplastic polyurethane (TPU). However, the anchor plates (118, 120, 122) could be made of another suitable material or combination of materials. In some implementations, an adhesive or ultrasonic welding are used to attach the anchor plates (118, 120, 122) to the underlying material of the arm sleeve 100.

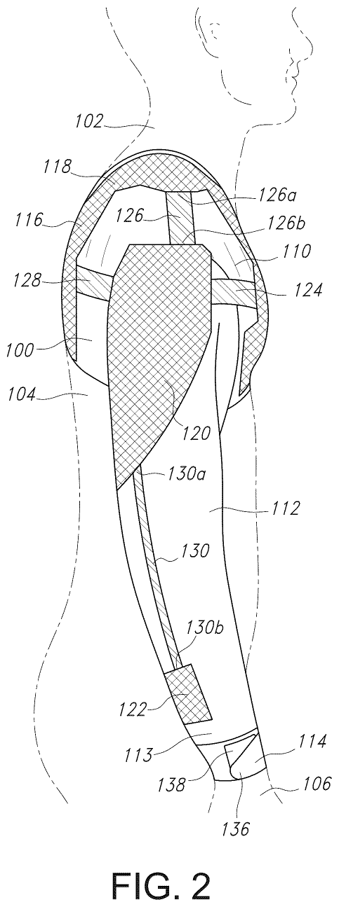

As shown in FIGS. 1 and 2, in some implementations, a first elastic band 124 of the framework 116 includes a first end 124a that extends from the portion of the anchor plate 118 overlaying the front side 110a of the torso portion 110 of the arm sleeve 100 to a second end 124b attached to the anchor plate 120 overlaying the triceps and deltoid of the throwing arm 106. The first end 124a is attached to an edge of the anchor plate 118 overlaying the front side 110a of the torso portion 110 and the second end 124b is attached to a forward edge of the anchor plate 120 overlaying the triceps and deltoid of the throwing arm 106. However, the first end 124a and/or the second end 124b of the first elastic band 124 could be attached to other areas of their respective anchor plates (118, 120).

The first elastic band 124 of the arm sleeve 100 is configured to play an active part throughout an overhead throwing motion. During the early cocking phase to the late cocking phase, the first elastic band 124 is stretched, storing elastic energy. During these phases of the throwing motion, the upper extremity of the user's throwing arm assumes the position of extension, abduction, external rotation, and horizontal abduction. As the upper extremity of the user's throwing arm moves during the early cocking phase and the late cocking phase, certain muscles stretch and thereby store elastic energy. Specifically, the internal rotators (e.g., the subscapularis, teres major, pectoralis major) and horizontal adductors (e.g., pectoralis major) of the user's shoulder. The elastic energy stored by the framework 116 of the arm sleeve 100, specifically the first elastic band 124, can now contribute to the acceleration of the overhead throwing motion by releasing stored elastic energy to assist the user's muscles during rotation and horizontal adduction of the shoulder.

As shown best in FIG. 2, in some implementations, a second elastic band 126 of the framework 116 includes a first end 126a that extends from a portion of the anchor plate 118 overlaying the shoulder of the throwing arm 106 to a second end 126b attached to the anchor plate 120 overlaying the triceps and deltoid of the throwing arm 106. The first end 126a is attached to an edge of the anchor plate 118 overlaying the shoulder of the throwing arm 106 and the second end 126b is attached to a top edge of the anchor plate 120 overlaying the triceps and deltoid of the throwing arm 106. However, the first end 126a and/or the second end 126b of the second elastic band 126 could be attached to other areas of their respective anchor plates (118, 120).

The second elastic band 126 of the arm sleeve 100 runs in the same direction as the supraspinatus and middle deltoid of the shoulder and is configured to decrease the concentric contraction force necessary for shoulder abduction during the cocking phase of a throwing motion. This is important because, fatigue of the supraspinatus muscle decreases the force present to properly couple the glenohumeral joint (the shoulder joint). Deltoid contraction will increase when the supraspinatus muscle is fatigued; this increase in deltoid contraction will superiorly translate the glenohumeral joint and thereby cause, or at least contribute to, shoulder impingement.

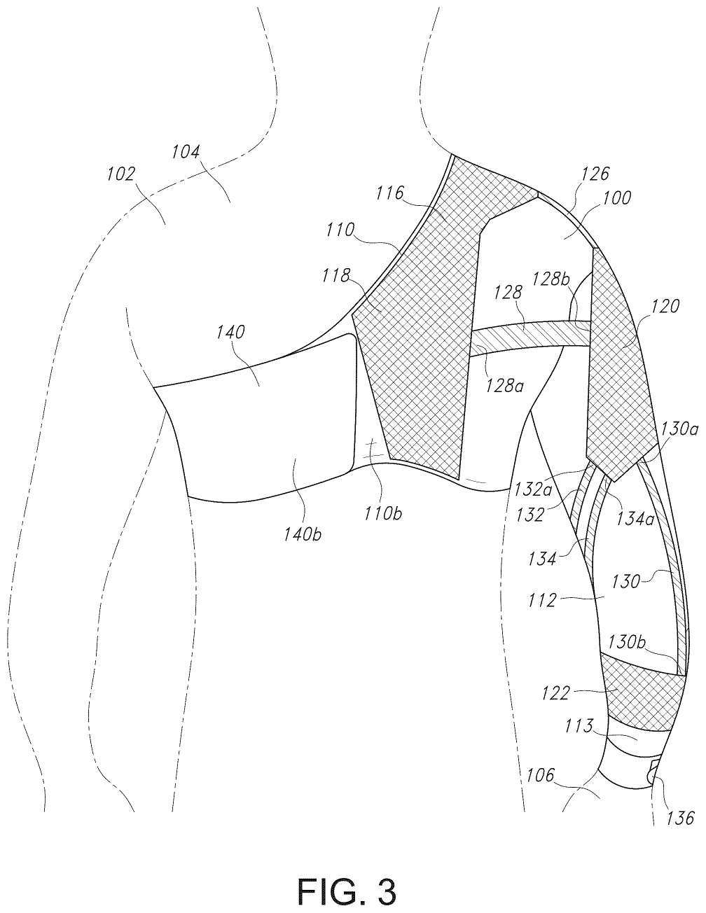

As shown in FIGS. 2 and 3, in some implementations, a third elastic band 128 of the framework 116 includes a first end 128a that extends from the portion of the anchor plate 118 overlaying the back side 110b of the torso portion 110 of the arm sleeve 100 to a second end 128b attached to the anchor plate 120 overlaying the triceps and deltoid of the throwing arm 106. The first end 128a is attached to an edge of the anchor plate 118 overlaying the back side 110b of the torso portion 110 and the second end 128b is attached to a rear edge of the anchor plate 120 overlaying the triceps and deltoid of the throwing arm 106. However, the first end 128a and/or the second end 128b of the third elastic band 128 could be attached to other areas of their respective anchor plates (118, 120).

The third elastic band 128 of the arm sleeve 100 is configured to assist eccentrically contracting muscles (e.g., posterior deltoid, teres minor, lower trapezius, infraspinatus, and rhomboids) during the deceleration phase of a throwing motion by preventing the upper extremity of the user's throwing arm from overextending. The third elastic band 128 stretches (or lengthens) as the upper extremity of the user's throwing arm moves into flexion and the shoulder rotates.

As shown in FIGS. 1-3, in some implementations, a fourth elastic band 130, a fifth elastic band 132, and a sixth elastic band 134 of the arm sleeve 100 each include a first end (130a, 132a, 134a) that extends from the anchor plate 120 overlaying the triceps and deltoid of the throwing arm 106 to a second end (130b, 132b, 134b) attached to the anchor plate 122 configured to partially encircle the forearm of the throwing arm 106. The fourth elastic band 130 is configured to extend from the anchor plate 120 across the triceps area of the arm, past the elbow joint, and posteriorly to the second end (see, e.g., FIG. 2). The fifth and sixth elastic bands (132, 134) are each configured to extend from the anchor plate 120, spiral posteriorly across the triceps area of the arm, past the elbow joint, and continues anteriorly to the second end (see, e.g., FIGS. 1 and 3).

As shown best in FIG. 2, the first end 130a of the fourth elastic band 130 is attached to a first bottom edge of the anchor plate 120 overlaying the triceps and deltoid of the throwing arm 106 and the second end 130b is attached to a top edge of the anchor plate 122 that partially encircles the forearm of the throwing arm 106. However, the first end 130a and/or the second end 130b of the fourth elastic band 130 could be attached to other areas of their respective anchor plates (120, 122).

As shown best in FIGS. 1 and 3, the first end 132a of the fifth elastic band 132 is attached to a second bottom edge of the anchor plate 120 overlaying the triceps and deltoid of the throwing arm 106 and the second end 132b is attached to the top edge of the anchor plate 122 that partially encircles the forearm of the throwing arm 106. However, the first end 132a and/or the second end 132b of the fifth elastic band 132 could be attached to other areas of their respective anchor plates (120, 122).

As shown best in FIGS. 1 and 3, the first end 134a of the sixth elastic band 134 is also attached to the second bottom edge of the anchor plate 120 overlaying the triceps and deltoid of the throwing arm 106 and the second end 134b is attached to the top edge of the anchor plate 122 that partially encircles the forearm of the throwing arm 106. However, the first end 134a and/or the second end 134b of the sixth elastic band 134 could be attached to other areas of their respective anchor plates (120, 122).

Together, the first elastic band 124, the fifth elastic band 132, and the sixth elastic band 134 are configured to minimize the amount of valgus torque and varus torque placed on the soft medial and lateral structures of the elbow joint.

Together, the first elastic band 124, fourth elastic band 130, fifth elastic band 132, and sixth elastic band 134 are configured to restrict the end-range external rotation of the shoulder at peak late cocking phase and thereby contribute to a decrease in distraction (medially) and compression (laterally) forces placed on the elbow joint. Also, the elastic band (124) that cross the glenohumeral joint acts as an active constraint for the shoulder and elbow during the late cocking phase of a throw.

Although not shown, in some implementations, the sixth elastic band 134 could be omitted, or a single elastic band configured to replace both the fifth elastic band 132 and the sixth elastic band 134 could be used.

In some implementations, the elastic bands (124, 126, 128, 130, 132, 134) of the framework 116 are made of an elastomeric material, such as silicone rubber. The tensile strength of the elastomer used to form the elastic bands (124, 126, 128, 130, 132, 134) of the framework 116 will affect (e.g., increase or decrease) the speed of a user's throw or swing. In some implementations, the elastic bands (124, 126, 128, 130, 132, 134) of the arm sleeve 100 may be made of an elastomeric material other than silicone rubber.

The elastic bands (124, 126, 128, 130, 132, 134) of the framework 116 may have any desired width, thickness, or elasticity. For example, the width, thickness, and elasticity of the elastic bands (124, 126, 128, 130, 132, 134) of the arm sleeve 100 may vary depending on the size, age, gender, extent of injury, etc. of the intended user. In some implementations, the elastic bands (124, 126, 128, 130, 132, 134) of the framework 116 may be made of differing materials, have differing thicknesses or width, may exhibit the same or different levels (moduli) of elasticity, etc. as desired.

Although not shown, in some implementations, each end (e.g., 124a, 124b) of an elastic band (e.g., 124) can be removably attached to their respective anchor plates (e.g., 118, 120). In this way, the tension provided by the band (e.g., 124) can be changed. This arrangement also allows the user to replace the band with another band that may be made of differing material(s), have differing thicknesses or width, may exhibit the same or different levels (moduli) of elasticity, etc. as desired. In some implementations, the first end and/or the second end of an elastic band may be attached to their respective anchor plates by a hook-and-loop fastening system. For example, hooks may be disposed on the first end 124a and/or the second end 124b of the first band 124 and loop pile fabric disposed on at least a portion of each anchor plate (118, 120).

As shown in FIGS. 1-5, the wrist strap 114 is attached to the distal end of the forearm segment 113 of the arm sleeve 100 and includes a tab 136 on its distal end. The wrist strap 114 is configured to wrap around the wrist of the user 102, and the tab 136 is configured to be removably affixed to the distal end of the forearm segment 113. In some implementations, hook-and-loop fasteners may be used to secured the tab 136 of the wrist strap 114 to the distal end of the forearm segment 113. For example, the tab 136 may include loop pile fabric thereon configured to catch on hooks 138 positioned adjacent the distal end of the arm portion 112. In use, the wrist strap 114 is wrapped about the wrist of the user 102 and, once the desired fit is achieved, the tab 136 is secured to the distal portion of the forearm segment 113 via the hook-and-loop fasteners. In this way, the user 102 can adjust the fit of the forearm segment 113 when donning the arm sleeve 100. In some implementations, other fasteners, or fastening mechanisms, such as snaps, buttons, etc. may be used to secure the tab 136 of the wrist strap 114 to the distal end of the forearm segment 113.

As shown in FIGS. 1 and 3, in some implementations, the arm sleeve 100 also includes a torso strap 140 used to position the torso portion 110 on the torso 104 of the user 102. The torso strap 140 extends between an attachment point 111a on the front side 110a of the torso portion 110 and an attachment point 111b on the back side 110b of the torso portion 110. When the arm sleeve 100 is being worn, the loop formed by the torso strap 140 is positioned between the user's arm and torso 104.

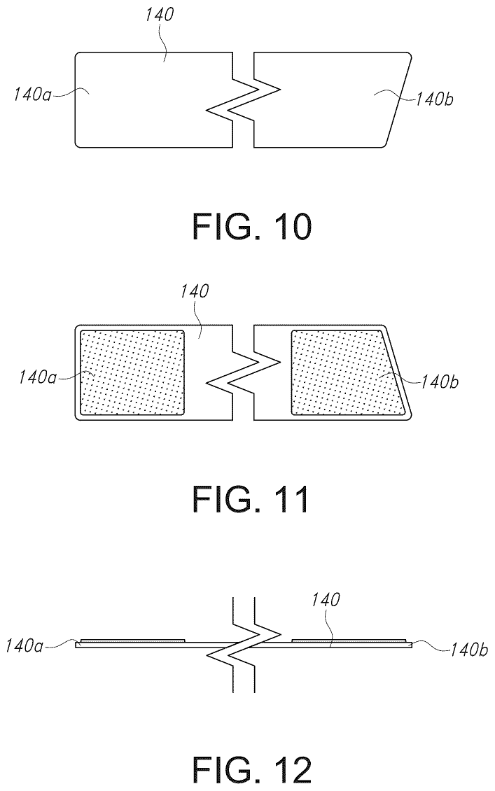

FIGS. 10-12 illustrate the torso strap 140 used to position the arm sleeve 100 on the torso 104 of the user 102, wherein the centrally located pair of adjacent lines are included for indicating that no particular length is being specified. Each end (140a, 140b) of the torso strap 140 can be secured to its corresponding attachment point (111a, 111b) through the use of hook-and-loop fasteners. In particular, the first end 140a and the second end 140b of the torso strap 140 each include hooks thereon that are configured to catch on loops of the attachment points (111a, 111b) on the torso portion 110. However, this arrangement could be changed so that the loops are on the ends (140a, 140b) of the torso strap 140 and the hooks are on the attachment points (111a, 111b) of the torso portion 110. The example torso strap 140 is made of an elastic nylon material. However, in some implementations, the torso strap 140 may be made of any suitable material that is selected in accordance with the implementations of the arm sleeve 100 disclosed herein.

Although not shown in the illustrations, in some implementations, the anchor plate 118 attached to the torso portion 110 of the arm sleeve 100 could be three separate anchor plates. In such implementations, one anchor plate would be positioned on the front side 110a of the torso portion 110, one anchor plate would be positioned to overlay the shoulder of the throwing arm 106, and one anchor plate would be positioned on the back side 110b of the torso portion 110.

The foregoing description of the invention is intended to be illustrative; it is not intended to be exhaustive or to limit the claims to the precise forms disclosed. Those skilled in the relevant art can appreciate that many modifications and variations are possible in light of the foregoing description and associated drawings.

Reference throughout this specification to an “embodiment” or “implementation” or words of similar import means that a particular described feature, structure, or characteristic is included in at least one embodiment of the present invention. Thus, the phrase “in some implementations” or a phrase of similar import in various places throughout this specification does not necessarily refer to the same embodiment.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

1. An arm sleeve, configured to provide external assistance to an arm and shoulder of a user during an arm motion, comprising:
a torso portion configured to be attached to the torso of the user;
an arm portion configured to cover at least a portion of the arm of the user; and
a framework comprising a plurality of anchor plates and elastic bands;
the plurality of anchor plates includes a first anchor plate, wherein the first anchor plate is attached to the torso portion and extends from a front side of the torso portion, across a top of the shoulder of the user, to a back side of the torso portion;
the plurality of anchor plates includes a second anchor plate, wherein the second anchor plate is attached to the arm portion and is positioned to overlay at least a portion of the triceps and deltoid of the arm of the user;
the plurality of anchor plates includes a third anchor plate, wherein the third anchor plate is attached to the arm portion and is positioned to partially encircle a forearm of the arm when the arm sleeve is being worn;
the elastic bands include a first elastic band with a first end that extends from a portion of the first anchor plate overlaying the front side of the torso portion to a second end attached to the second anchor plate;
the elastic bands include a second elastic band with a first end that extends from a portion of the first anchor plate overlaying the shoulder of the user to a second end attached to the second anchor plate;
the elastic bands include a third elastic band with a first end that extends from a portion of the first anchor plate overlaying the back side of the torso portion to a second end attached to the second anchor plate;
the elastic bands include a fourth elastic band with a first end that extends from the second anchor plate across the triceps area of the arm, past the elbow joint, and posteriorly to a second end that is attached to the third anchor plate; and
the elastic bands include a fifth elastic band with a first end that extends from the second anchor plate, spirals posteriorly across the triceps area of the arm, past the elbow joint, and continues anteriorly to a second end that is attached to the third anchor plate;
wherein the first anchor plate, the second anchor plate, and the third anchor plate each serve as anchor points for one or more of the elastic bands of the framework.

2. The arm sleeve of claim 1, wherein the framework of the arm sleeve is operable to provide assistance to at least one muscle supporting an ulnar collateral ligament (UCL) of the arm of the user and to at least one muscle supporting the glenohumeral joint of the shoulder of the user during the arm motion.

3. The arm sleeve of claim 1, wherein the first anchor plate, the second anchor plate, and the third anchor plate are each made of a rigid material and configured to substantially conform to the general area of the torso or arm that they overlay.

4. The arm sleeve of claim 1, wherein each of the first elastic band, the second elastic band, and the third elastic band extends separately from the first anchor plate.

5. The arm sleeve of claim 4, wherein each of the fourth elastic band and the fifth elastic band extends separately from the second anchor plate.

6. The arm sleeve of claim 1, wherein the arm portion includes a strap configured to secure a distal end of the arm portion to a portion of the arm of the user.

7. The arm sleeve of claim 1, further comprising a strap configured to position the torso portion of the arm sleeve on the torso of the user, the strap extends between an attachment point on the front side and an attachment point on the back side of the torso portion.

8. An arm sleeve, configured to provide external assistance to an arm and shoulder of a user during an arm motion, comprising:
a torso portion configured to be attached to the torso of the user;
an arm portion configured to cover at least a portion of the arm of the user; and
a framework comprising a plurality of anchor plates and elastic bands;
the plurality of anchor plates includes a first anchor plate, wherein the first anchor plate is attached to the torso portion and extends from a front side of the torso portion, across a top of the shoulder of the user, to a back side of the torso portion;
the plurality of anchor plates includes a second anchor plate, wherein the second anchor plate is attached to the arm portion and is positioned to overlay at least a portion of the triceps and deltoid of the arm of the user;
the plurality of anchor plates includes a third anchor plate, wherein the third anchor plate is attached to the arm portion and is positioned to partially encircle a forearm of the arm when the arm sleeve is being worn;
the elastic bands include a first elastic band with a first end that extends from a portion of the first anchor plate overlaying the front side of the torso portion to a second end attached to the second anchor plate;
the elastic bands include a second elastic band with a first end that extends from a portion of the first anchor plate overlaying the shoulder of the user to a second end attached to the second anchor plate;
the elastic bands include a third elastic band with a first end that extends from a portion of the first anchor plate overlaying the back side of the torso portion to a second end attached to the second anchor plate;
the elastic bands include a fourth elastic band with a first end that extends from the second anchor plate across the triceps area of the arm, past the elbow joint, and posteriorly to a second end that is attached to the third anchor plate;
the elastic bands include a fifth elastic band with a first end that extends from the second anchor plate, spirals posteriorly across the triceps area of the arm, past the elbow joint, and continues anteriorly to a second end that is attached to the third anchor plate; and
the elastic bands include a sixth elastic band with a first end that extends from the second anchor plate, spirals posteriorly across the triceps area of the arm, past the elbow joint, and continues anteriorly to a second end that is attached to the third anchor plate;
wherein the first anchor plate, the second anchor plate, and the third anchor plate each serve as anchor points for one or more of the elastic bands of the framework.

9. The arm sleeve of claim 8, wherein the framework of the arm sleeve is operable to provide assistance to at least one muscle supporting an ulnar collateral ligament (UCL) of the arm of the user and to at least one muscle supporting the glenohumeral joint of the shoulder of the user during the arm motion.

10. The arm sleeve of claim 8, wherein the first anchor plate, the second anchor plate, and the third anchor plate are each made of a rigid material and configured to substantially conform to the general area of the torso or arm that they overlay.

11. The arm sleeve of claim 8, wherein each of the first elastic band, the second elastic band, and the third elastic band extends separately from the first anchor plate.

12. The arm sleeve of claim 11, wherein each of the fourth elastic band, the fifth elastic band, and the sixth elastic band extends separately from the second anchor plate.

13. The arm sleeve of claim 8, wherein the arm portion includes a strap configured to secure a distal end of the arm portion to a portion of the arm of the user.

14. The arm sleeve of claim 8, further comprising a strap configured to position the torso portion of the arm sleeve on the torso of the user, the strap extends between an attachment point on the front side and an attachment point on the back side of the torso portion.
